(19) United States
(12) Patent Application Publication
(10) Pub. No.: US 2003/0120342 A1
(43) Pub. Date: Jun. 26, 2003

(54) Intraocular lens

(76) Inventor: George F. Green, Victor, NY (US)

Correspondence Address: Bausch & Lomb Incorporated, One Bausch & Lomb Place, Rochester, NY, 14604-2701, US

(21) Appl. No.: 10/027,376

(22) Filed: Dec. 21, 2001

(51) Int. Cl. A61F 2/16

(52) U.S. Cl. 623/6.16

(57) ABSTRACT

An intraocular lens for inhibiting posterior capsular opacification, or secondary cataract, includes an optic having a peripheral wall provided with a sharp posterior edge wherein a majority of the optic mass is located posteriorly of the optic-haptic junction.

### BACKGROUND OF THE INVENTION

The present invention relates to intraocular lenses (IOLs) for implantation in an aphakic eye where the natural lens has been removed due to damage or disease (e.g., a cataractous lens). The present invention more particularly relates to a novel IOL designed to inhibit the unwanted growth of lens epithelial cells (LECs) between the IOL and posterior capsular bag, also known as posterior capsule opacification or “PCO” to those skilled in the art.

A common and desirable method of treating a cataract eye is to remove the clouded, natural lens and replace it with an artificial IOL in a surgical procedure known as cataract extraction. In the extracapsular extraction method, the natural lens is removed from the capsular bag while leaving the posterior part of the capsular bag (and preferably at least part of the anterior part of the capsular bag) in place within the eye. In this instance, the capsular bag remains anchored to the eye's ciliary body through the zonular fibers. In an alternate procedure known as intracapsular extraction, both the lens and capsular bag are removed in their entirety by severing the zonular fibers and replaced with an IOL which must be anchored within the eye absent the capsular bag. The intracapsular extraction method is considered less attractive as compared to the extracapsular extraction method since in the extracapsular method, the capsular bag remains attached to the eye's ciliary body and thus provides a natural centering and locating means for the IOL within the eye. The capsular bag also continues its function of providing a natural barrier between the aqueous humor at the front of the eye and the vitreous humor at the rear of the eye.

One known problem with extracapsular cataract extraction is posterior capsule opacification, or secondary cataract, where proliferation and migration of lens epithelial cells occur along the posterior capsule behind the IOL posterior surface which creates an opacification of the capsule along the optical axis. This requires subsequent surgery, such as an Er:YAG laser capsulotomy, to open the posterior capsule and thereby clear the optical axis. Undesirable complications may follow the capsulotomy. For example, since the posterior capsule provides a natural barrier between the back of the eye vitreous humor and front of the eye aqueous humor, removal of the posterior capsule allows the vitreous humor to migrate into the aqueous humor which can result in serious, sight-threatening complications. It is therefore highly desirable to prevent posterior capsule opacification in the first place and thereby obviate the need for a subsequent posterior capsulotomy.

Various methods have been proposed in the art to prevent or at least minimize PCO and thus also the number of Er:YAG laser capsultomies required as a result of PCO. These PCO prevention methods include two main categories: mechanical means and pharmaceutical means.

In the mechanical means category of PCO prevention, efforts have been directed at creating a sharp, discontinuous bend in the posterior capsule wall which is widely recognized by those skilled in the art as an effective method for minimizing PCO. See, for example, Posterior Capsule Opacification by Nishi, Journal of Cataract & Refractive Surgery, Vol. 25, January 1999. This discontinuous bend in the posterior capsule wall can be created using an IOL having a posterior edge which forms a sharp edge with the peripheral wall of the IOL.

In the pharmaceutical means of PCO prevention, it has been proposed to eliminate LEC and/or inhibit LEC mitosis by using an LEC-targeted pharmaceutical agent. See, for example, U.S. Pat. No. 5,620,013 to Bretton entitled “Method For Destroying Residual Lens Epithelial Cells”. While this approach is logical in theory, putting such a method into clinical practice is difficult due to complications arising, for example, from the toxicity of some of the LEC inhibiting agents themselves (e.g., saporin), as well as the difficulty in ensuring a total kill of all LECs in the capsular bag. Any remaining LECs may eventually multiply and migrate over the IOL, eventually resulting in PCO despite the attempt at LEC removal at the time of surgery.

By far the most promising method for inhibiting LEC formation on the posterior surface of an IOL is the mechanical means, i.e., by designing the IOL to have a sharp peripheral edge particularly at the posterior surface—peripheral edge juncture to create a discontinuous bend in the posterior capsule wall. This discontinuous bend in the posterior capsule wall has been clinically proven to inhibit the growth and migration of LECs past this bend and along the IOL surface. One of the early reports of this PCO-inhibiting effect of a planoconvex IOL may be found in Explanation of Endocapsule Posterior Chamber Lens After Spontaneous Posterior Dislocation by Nishi et al, J Cataract & Refractive Surgery-Vol 22, March 1996 at page 273 wherein the authors examined an explanated planoconvex PMMA IOL where the posterior surface of the IOL was planar and formed a square edge with the peripheral edge of the IOL:

“Macroscopic view of the explanted IOL and capsule revealed a 9.5 mm capsule diameter. The open circular loops fit well along the capsule equator. The capsule equator not in contact with the haptic was also well maintained (FIG. 3). An opaque lens mass (Soemmering's ring cataract) was seen between the haptics and optic. The posterior capsule facing the IOL optic was clear.

Histopathological examination of the explanted capsule revealed few epithelial cells (LECs) on the posterior capsule. Between the loops and the optic, a lens mass with accumulation at the edge of the optic was seen (FIG. 4). There was an obvious bend in the posterior capsule at this site.” (Emphasis added.)

Thus, in the years since this report, the industry has seen much activity on creating IOLs with sharp posterior edges so as to create a sharp, discontinuous bend in the posterior capsule wall. While IOLs having a sharp posterior edge have proven to inhibit PCO compared to IOLs having rounded edges at the posterior surface-peripheral edge juncture, there still remains the possibility of LECs migrating along the posterior capsule and behind the IOL surface, especially if there is uneven contact and force of the IOL periphery with the capsular bag. This may happen, for example, should the IOL move within the capsular bag following surgery. There therefore remains a need for an improved IOL design which addresses the problem of LEC migration and subsequent PCO formation despite having an IOL with a sharp posterior edge.

### SUMMARY OF THE INVENTION

The present invention addresses the problem of PCO by providing a single-piece IOL having a sharp peripheral edge wherein a majority of the optic mass is located posteriorly of the optic-haptic juncture. This design is an improvement over other single square edge IOL designs in that by shifting the optic mass posteriorly of the optic-haptic junction, the in-vivo posterior vault is improved and creates a deeper discontinous bend in the posterior capsular wall. The present IOL design is also relatively easy to manufacture compared with other, more complicated IOL periphery designs which have been proposed in the prior art for inhibiting LEC migration. See, for example, the following patents and publications which show various IOL optic periphery designs:

U.S. Pat. No. 5,171,320 issued to Nishi on Dec. 15, 1992

U.S. Pat. No. 5,693,093 issued to Woffinden et al on Dec. 2, 1997

U.S. Pat. No. 6,162,249 issued to Deacon et al on Dec. 19, 2000

### BRIEF DESCRIPTION OF THE DRAWING

FIG. 1 is a cross-sectional view of a human eye showing the natural lens within the capsular bag of the eye;

FIG. 2 is a cross-sectional view of a human eye showing the natural lens removed and replaced with a prior art IOL;

FIG. 3 is an elevational view of the inventive IOL with the haptics shown fragmented;

FIG. 4 is an enlarged, fragmented, cross-sectional view showing the detail of the peripheral wall configuration of the IOL of the present invention; and

FIG. 5 is an elevational view of a prior art IOL with the haptics shown fragemented.

### DETAILED DESCRIPTION

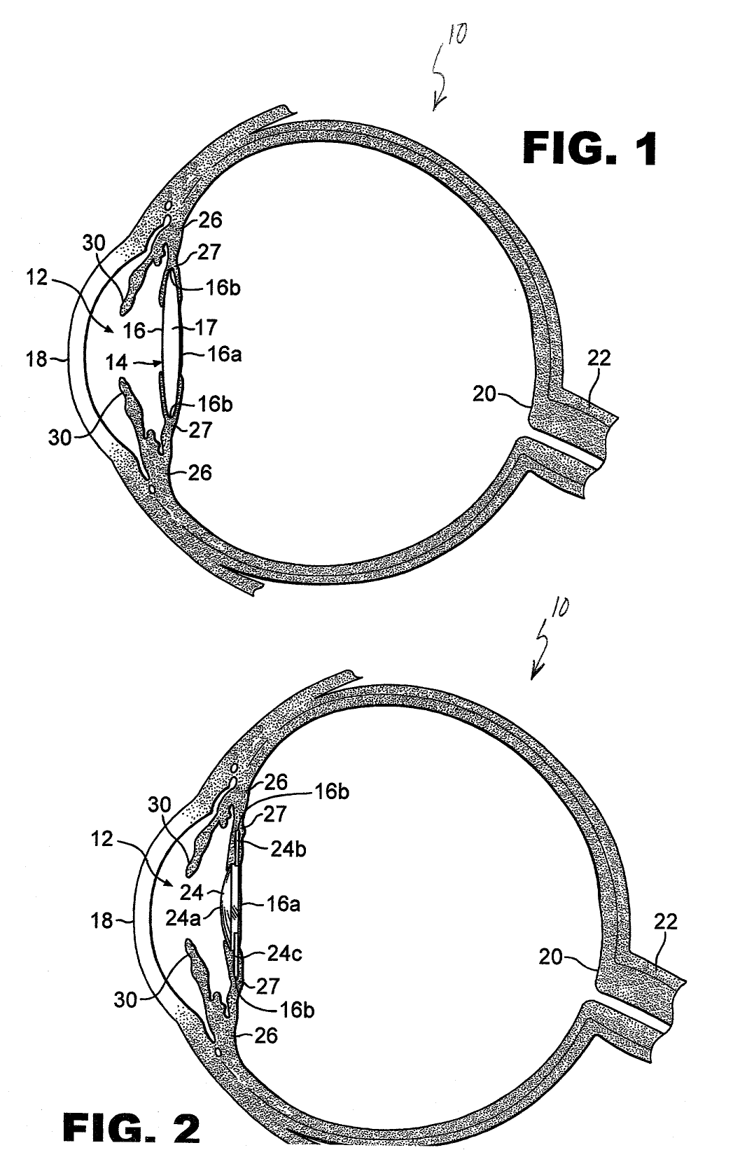

Referring now to the drawing, there is seen in FIG. 1 a cross-sectional view of a human eye 10 having an anterior chamber 12 and a posterior chamber 14 separated by the iris 30. Within the posterior chamber 14 is a capsule 16 which holds the eye's natural crystalline lens 17. Light enters the eye by passing through the cornea 18 to the crystalline lens 17 which act together to direct and focus the light upon the retina 20 located at the back of the eye. The retina connects to the optic nerve 22 which transmits the image received by the retina to the brain for interpretation of the image.

In an eye where the natural crystalline lens has been damaged (e.g., clouded by cataracts), the natural lens is no longer able to properly focus and direct incoming light to the retina and images become blurred. A well known surgical technique to remedy this situation involves removal of the damaged crystalline lens which may be replaced with an artificial lens known as an intraocular lens or IOL such as prior art IOL 24 seen in FIG. 2. Although there are many different IOL designs as well as many different options as to exact placement of an IOL within an eye, the present invention concerns itself with an IOL for implanting inside the substantially ovoid-shaped capsule 16 of eye 10. This implantation technique is commonly referred to in the art as the “in-the-bag” technique. In this surgical technique, a part of the anterior portion of the capsular bag is cut away (termed a “capsularhexis”) while leaving the posterior capsule 16a intact and still secured to the ciliary body 26.

Thus, in the “in-the-bag” technique of IOL surgery, the IOL is placed inside the capsule 16 which is located behind the iris 30 in the posterior chamber 14 of the eye. An IOL includes a central optic portion 24a which simulates the extracted natural lens by directing and focusing light upon the retina, and further includes a means for securing the optic in proper position within the capsular bag. A common IOL structure for securing the optic is called a haptic which is a resilient structure extending radially outwardly from the periphery of the optic. In a particularly common IOL design, two haptics 24b, 24c extend from opposite sides of the optic and curve to provide a biasing force against the inside of the capsule which secures the optic in the proper position within the capsule (see FIG. 2).

As stated in the Background section hereof, an undesirable post-surgical condition known as posterior capsule opacification or PCO may occur which results in an implanted IOL becoming clouded and thus no longer able to properly direct and focus light therethrough. The main cause for this condition is the mitosis and migration of lens epithelial cells (LECs) across the posterior surface of the capsule behind the IOL optic. As seen in FIG. 2, the posterior surface 16a of the capsule 16 touches the posterior surface of the IOL optic 24a. When the damaged natural lens is surgically removed, a number of LECs may remain within the capsule 16, particularly at the equator 16b thereof which is the principle source of germinal LECs. Although a surgeon may attempt to remove all LECs from the capsular bag at the time of IOL implantation surgery, it is nearly impossible to remove every single LEC. Any remaining LECs can multiply and migrate along the posterior capsule wall 16a. This is especially true in IOLs having rounded edges, where it has been found that clinically significant PCO results in about 20%-50% of patients three years post surgery. A presently popular and effective method of preventing PCO is to create a sharp, discontinuous bend in the posterior capsule wall 16a as explained in the Background section hereof

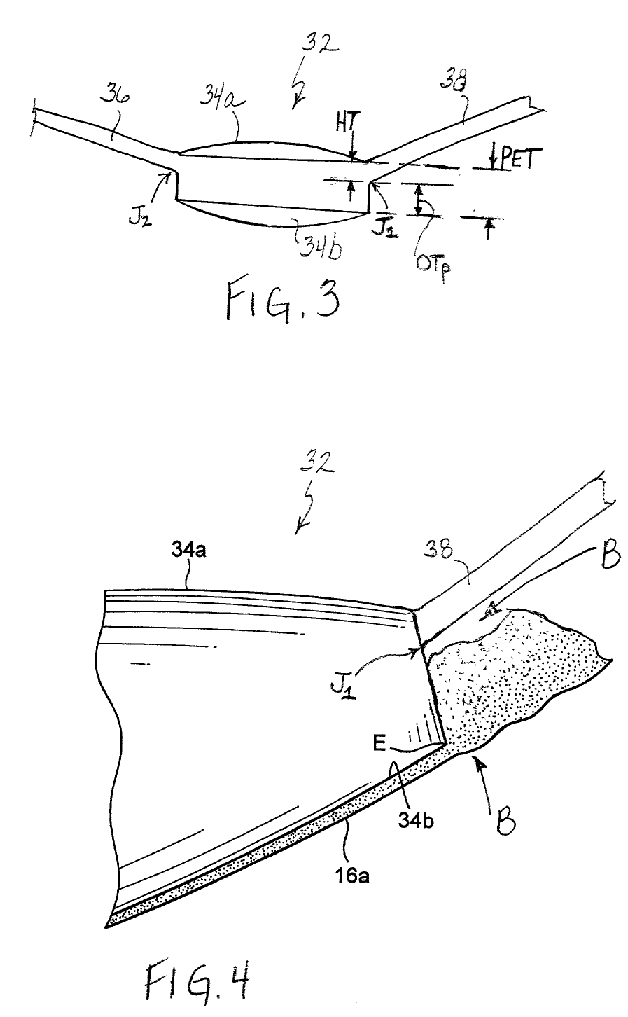

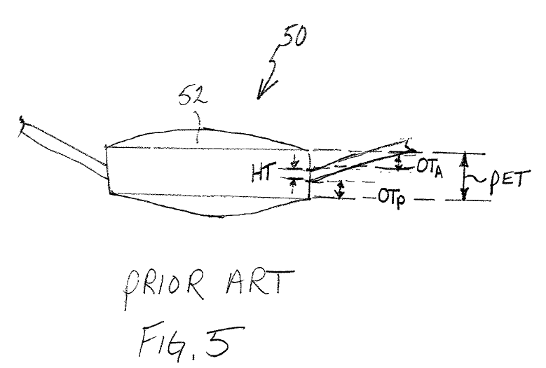

Referring now to FIGS. 3 and 4, the inventive IOL 32 is shown. IOL 32 is seen to include a central optic portion 34 having opposite anterior and posterior surfaces 34a and 34b, respectively, defined by a peripheral wall P. When implanted within the eye, anterior optic surface 34a faces the cornea 18 and posterior optic surface 34b faces the retina 20. A pair of haptics 36,38 are attached to and extend from opposite sides of the peripheral wall P of optic portion 34 and are configured to provide a biasing force against the interior of the capsule 16 to properly position IOL 32 therein. More particularly, the haptics 36,38 are configured such that upon implanting the IOL with the capsular bag, the haptics engage the interior surface of the capsular bag. The engagement between the haptics and capsule creates a biasing force causing the implanted IOL optic 34 to vault posteriorly toward the retina 20 whereupon the posterior surface 34b of the IOL optic presses tightly against the interior of the posterior capsule wall 16a of capsule 16. It is noted that other known IOL positioning means (e.g., plate haptics) are possible and within the scope of the invention. Furthermore, IOL 32 may be made from any suitable IOL material, e.g., PMMA, silicone, hydrogels and composites thereof

Referring still to FIGS. 3 and 4, it is seen that IOL optic peripheral wall P includes a sharp posterior edge E defined at the juncture of posterior surface 34b and peripheral wall P. With the haptics 36,38 providing the biasing force explained above, the optic posterior surface 34b will press tightly against the posterior capsule wall 16a. Since capsule 16 is somewhat resilient in nature, the force of the IOL optic against the capsule wall results in the IOL indenting into the posterior capsule wall. The sharp edge E of the IOL optic thus forcibly indents into the capsule wall and thereby creates a discontinuous bend in the posterior capsule wall at this point as indicated at arrow B in FIG. 4. As explained above, this discontinuous bend B in the posterior capsule wall 16a acts to inhibit LEC migration past this point (i.e., between the posterior capsule wall 16a and IOL posterior surface 34b) and PCO is inhibited.

As stated above, the present IOL is an improvement over prior IOLs having a sharp posterior edge in that the majority (i.e., greater than 50%) of the optic mass is located posteriorly of the optic-haptic junctures J₁ and J₂. In prior art IOLs, such as IOL 50 seen in FIG. 5, a common peripheral edge thickness “PET” for a lens is about 400 μm with the haptic thickness “HT” being about 150 μm and joined at the optic 52 at the center of the peripheral wall 54 thereof This leaves about 125 μm in anterior optic thickness “OT_{A}” and posterior optic thickness “OT_{P}” on either side of the optic-haptic junction. Realizing that the degree to which the optic periphery is able to indent into the capsule wall is limited by the optic-haptic juncture, the prior art IOLs are limited to a capsular indentation of about 125 μm (i.e., the distance of posterior optic thickness “OT_{P}”). Conversely, in the embodiment of a comparable IOL lens as shown herein having essentially the same peripheral edge thickness “PET” of about 400 μm and haptic thickness“HT” of about 125 μm with, in accordance with the present invention, the optichaptic junction positioned adjacent anterior optic surface 34a such that more than half of the optic mass is located posteriorly of the optic-haptic junction, the posterior optic thickness “OT_{P}” in the inventive IOL is about 130 to about 250 μm, and more preferably about 150-250 μm, and most preferably about 250 μm, while the anterior optic thickness “OT_{A}” is about 120 μm to about zero μm, and more preferably about 100 μm to about zero μm, and most preferably about zero μm.

It will thus be appreciated that the inventive IOL allows its posterior peripheral edge to indent the capsular wall to a greater degree, specifically, in the example provided herein, an indentation up to 250 μm rather than the 150 μm indentation allowed by a comparable prior art IOL. In a patient where the optic indents into the posterior capsule as seen in FIG. 4, once LECs begin migrating and reach the IOL optic 34, they will encounter sharp bend B in the capsule formed by IOL sharp edge E. As discussed above, the inventive IOL provides a deeper, more complex frill formation in the posterior capsule wall than IOL designs of the prior art and thus provides an improved barrier against migrating LECs.

It is noted that other lens dimensions (i.e., peripheral edge thickness and optic thickness), as well as other designs and curvatures may be employed with the present invention (e.g., plano-convex, plano-concave, biconcave, ashpere, toric, multifocal, accomodating, etc.) so long as the majority of the optic mass is located posteriorly of the optic-haptic juncture to realize the above-described effects and benefits of the present invention.

Methods which may be employed to form the IOL include lathing and molding, for example. It is also preferred that IOL 32 undergo tumble polishing either prior to forming the sharp posterior edge, or with the sharp posterior edge masked so as to ensure edge E retains its sharpness.

What is claimed is:

1. An intraocular lens for implanting in a human eye, comprising:
a) a lens optic defined by opposite anterior and posterior surfaces and a peripheral wall having a peripheral edge thickness “PET”, the juncture of said posterior surface and said peripheral wall defining a sharp edge; and
b) at least one haptic attached to and extending from said peripheral wall, said at least one haptic having a haptic thickness“HT” and adapted to apply a biasing force against said optic in the direction of said posterior optic surface upon implanting said intraocular lens in said human eye, the juncture of said haptic and said peripheral wall defining a posterior optic thickness “OTp” and an anterior optic thickness “OTa” on either side of said haptic, and wherein said posterior optic thickness “OTp” is more than half of said peripheral edge thickness “PET”.

2. The intraocular lens of claim 1, wherein said peripheral edge thickness “PET” is about 400 μm and said haptic thickness“HT” is about 125 μm, and said posterior optic thickness “OT_{P}” is about 130 to about 250 μm and said anterior optic thickness “OT_{A}” is about 120 μm to about zero μm.

3. The intraocular lens of claim 2, wherein said posterior optic thickness “OT_{P}” is about 150-250 μm and said anterior optic thickness “OT_{A}” is about 100 μm to about zero μm.

4. The intraocular lens of claim 2, wherein said posterior optic thickness “OT_{P}” is about 250 μm, and said anterior optic thickness “OT_{A}” is about zero μm.
